(19) United States
(12) Patent Application Publication
(10) Pub. No.: US 2025/0354976 A1
(43) Pub. Date: Nov. 20, 2025

(54) AIRWAY ADAPTER HAVING LIQUID CONTAINMENT STRUCTURES

(71) Applicant: GE Precision Healthcare LLC, Waukesha, WI (US)

(72) Inventors: Mika Hietala, Espoo (FI); Asko Saarelainen, Espoo (FI); Jussi Pitkonen, Espoo (FI)

(21) Appl. No.: 18/666,425

(22) Filed: May 16, 2024

(51) Int. Cl. G01N 33/497; A61M 16/08

(52) U.S. Cl. CPC G01N 33/497; A61M 16/085

(57) ABSTRACT

An airway adapter for gas measurement by a mainstream gas analyzer, the airway adapter including a body configured to connect in-series with a ventilation circuit, the body including an inner surface providing a conduit which extends through the body to provide a flow path for the ventilation gas to pass between a first end and a second end of the adapter, wherein opposing sides of a central portion of the inner surface protrude inward to provide a narrowed section, the narrowed section comprising an upper measurement chamber and a lower fluid channel; and at least two windows provided in the body at the measurement chamber, the at least two windows configured to pass radiation for measuring gas within the measurement chamber. The inner surface of the airway adapter is configured to direct fluid away from the measurement chamber and into the fluid channel.

### FIELD

The present disclosure generally relates to respiratory gas sensor systems that measure one or more respiratory gas components in a breathing circuit of a patient, and more particularly to a sensor adapter which directs fluids away from the sensor optics.

### BACKGROUND

In anesthesia and in intensive care, the condition of a patient is often monitored by analyzing the gas inhaled and exhaled by the patient for its content. For this reason, either a small portion of the respiratory gas is delivered to a gas analyzer, or the gas analyzer is directly connected to the respiratory circuit. In a non-dispersive infrared (NDIR) gas analyzer, the measurement is based on the absorption of infrared (IR) radiation in the gas sample. A radiation source directs a beam of infrared radiation through a measuring chamber to a radiation detector whose output signal depends on the strength of the absorption of the radiation in the sample gas.

The radiation source typically comprises an electrically heated filament or surface area and radiation collecting optics and emits radiation within a spectral region. The gas sample to be analyzed is fed through the measuring chamber. The measuring chamber can be a tubular space, for example, with inlet and outlet for the sample gas and provided with windows that have high transmission at the measurement IR wavelength and permit transmission of the IR wavelength through the chamber. Radiation is absorbed by the gas sample when passing through the measuring chamber, and thus the amount of the measurement IR wavelength that is transmitted through the chamber (i.e., from one window to the other) is indicative of certain gas component amount(s) in the gas sample. However, liquid in the measuring chamber may interact with the IR radiation in a way that decreases accuracy of the sensor.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

According to an embodiment, an airway adapter for gas measurement by a mainstream gas analyzer may include a body configured to connect in-series with a ventilation circuit carrying ventilation gas to and from a patient. The body may include a first end, a second end, a top, and a bottom; an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and at least two windows provided in the body at the measurement chamber, the at least two windows configured to pass radiation for measuring gas within the measurement chamber. The inner surface of the airway adapter may be configured to direct fluid away from the measurement chamber and into fluid channel.

According to an embodiment, a ventilation system may include a ventilator configured to ventilate a patient; and a ventilation circuit configured to carry ventilation gas from the ventilator to a patient and from the patient to the ventilator, the ventilation circuit comprising and airway adapter configured to connect in-series in the ventilation circuit. The airway adapter may include a body: a first end, a second end, a top, and a bottom, an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and at least two windows provided in the body at the measurement chamber, the at least two windows configured to pass radiation for measuring gas within the measurement chamber. The inner surface of the airway adapter may be configured to direct fluid away from the measurement chamber and into fluid channel.

Various other features, objects, and advantages of the invention will be made apparent from the following description taken together with the drawings.

### BRIEF DESCRIPTION OF THE DRAWINGS

The present disclosure is described with reference to the following Figures.

FIG. 1A shows a ventilation system, according to an embodiment.

FIG. 1B shows a gas analyzer attached to an airway adapter, according to an embodiment.

FIGS. 2A and 2B are cross-sectional views of the airway adapter taken along cross-section line A in FIG. 3B, according to an embodiment.

FIG. 2C is a cross-sectional view of the airway adapter taken along cross-section line B in FIG. 3A, according to an embodiment.

FIG. 3A is a side view of the airway adapter, according to an embodiment.

FIG. 3B is a ventilator end view of the airway adapter, according to an embodiment.

FIG. 3C is an angled view of a ventilator end of the airway adapter, according to an embodiment.

FIG. 3D is a patient end view of the airway adapter, according to an embodiment.

FIG. 4A is a patient end view of the airway adapter at an X angle of zero and a Y angle of zero with accumulated liquid, according to an embodiment.

FIG. 4B is a patient end view of the airway adapter at an X angle of 0 and a Y angle of 90 with accumulated liquid, according to an embodiment.

FIG. 5A is a cross-sectional view of the airway adapter, taken along cross-section line B, at an X angle of zero and a Y angle of zero with accumulated liquid, according to an embodiment.

FIG. 5B is a cross-sectional view of the airway adapter, taken along cross-section line B, at an X angle of zero and a Y angle of 45 with accumulated liquid, according to an embodiment.

FIG. 5C is a cross-sectional view of the airway adapter, taken along cross-section line B, at an X angle of zero and a Y angle of 60 with accumulated liquid, according to an embodiment.

FIG. 6A is a cross-sectional view of the airway adapter, taken along cross-section line A, at an X angle of zero and a Y angle of zero with accumulated liquid, according to an embodiment.

FIG. 6B is a cross-sectional view of the airway adapter, taken along cross-section line B, at an X angle of 45 and a Y angle of 0 with fluid flow lines, according to an embodiment.

FIG. 6C is a cross-sectional view of the airway adapter, taken along cross-section line B, at an X angle of 90 and a Y angle of 0 with fluid flow lines, according to an embodiment.

### DETAILED DESCRIPTION

The present inventor has recognized that a problem with existing airway adapter s, or cuvettes, for facilitating gas measurement by a mainstream gas analyzer is that liquids and vapors collect inside the measurement chamber of the airway adapter. The liquids accumulate on the windows of the airway adapter through which the gas analyzer makes measurements such that accurate measurements cannot be performed by the gas analyzer. This problem increases as the airway adapter is moved away from an ideal orientation. Ideally, the fluid path of the airway adapter is oriented at a 10-50 degrees angle with respect to the ground (e.g. 40-80 degrees with respect to gravity). This angle provides the necessary slope for any liquids to drain out of the adapter without pooling while also directing any liquids to a bottom of the adapter (e.g., away from the windows). In cases where the flow path of the airway adapter approaches an orientation parallel to the ground, fluid drainage will decrease resulting in fluid pooling in the airway adapter. In cases where the flow path of the adapter approaches an orientation perpendicular to the ground, the fluid will not be directed to a bottom of the adapter.

In view of the foregoing problems and challenges, the inventor developed the disclosed airway adapter having features to direct liquids away from a gas path which includes the measurement chamber having the windows through which the gas analyzer makes measurements. The measurement chamber is configured to allow the ventilation gas to pass the windows for accurate measurement from the gas analyzer. The airway adapter includes a liquid path separated from a gas path, wherein the liquid path is configured to contain liquid away from the gas path to isolate as much of the accumulated liquid as possible away from the measurement chamber and particularly the optical windows. Thus, the one or more features are provided such that most, or as much as possible, of the liquid flows through the secondary path, or is otherwise contained in the secondary path away from the measurement chamber.

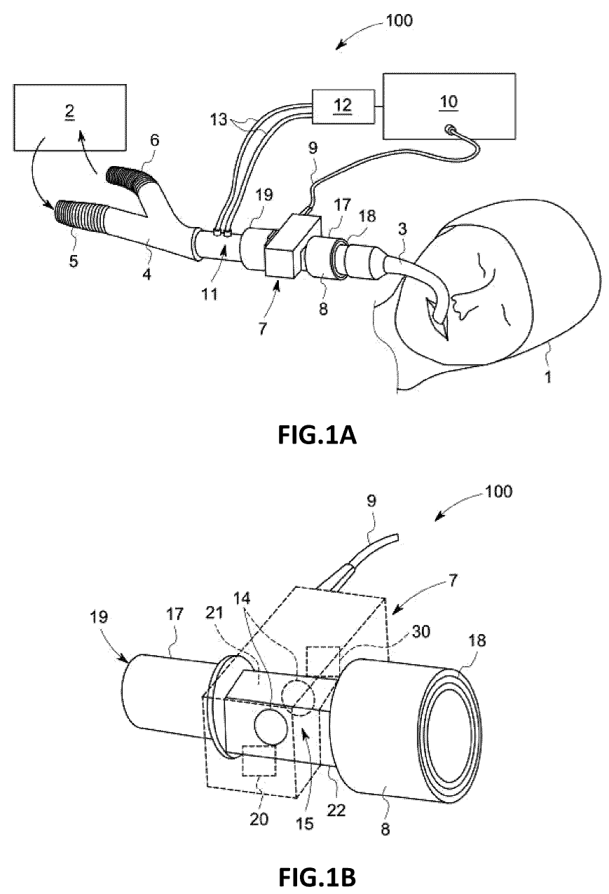

Exemplary embodiments of the disclosed airway adapter and system comprising the disclosed airway adapter are shown in FIGS. 1A-6C and variously discussed herein.

FIG. 1A shows a ventilation system, according to an embodiment. FIG. 1B shows a gas analyzer attached to an airway adapter, according to an embodiment. As shown in FIGS. 1A and 1B, the disclosed respiratory gas sensor system 100 includes an airway adapter 8, or cuvette, with a secondary path configured to contain liquids that have accumulated in that area of the ventilation circuit away from the gas path where the gas analyzer 7 is conducting measurements. The airway adapter 8 has body 17 with a top side 21 and a bottom side 22. The system 100 is generally configured such that the bottom side 22 is below the top side 21 such that gravity forces liquids downward toward the bottom side 22. The body 17 has a first end 18 and a second end 19, each end configured to attach to a respective element within the ventilation circuit. In the depicted example, the first end 18 on the patient-side of the body 17 is configured to connect to an endotracheal tube 3 and the second end 19 is configured to connect to spirometry adapter 11 and/or to a Y-piece 4 that circulates gas to and from the ventilator 2.

The gas analyzer 7 may removably connect to the airway adapter 8, such as by clips on the airway adapter 8 configured to create a friction connection thereto. The top side 21 may be configured to connect to the gas analyzer 7. For example, the adapter body 17 may include two opposing clips configured to removably connect to the airway adapter, which is positioned over the top side 21 of the center portion of the airway adapter 8 and extends over the sides of the center portion so as to conduct gas measurements through the windows 14.

As exemplified in FIG. 1A, a ventilation circuit with a medical gas analyzer is shown. A patient 1 is connected to a ventilator 2 using an endotracheal tube 3, a Y-piece 4, an inspiratory limb 5, and an expiratory limb 6. A gas analyzer 7 is connected to an airway adapter 8, which is connected to the intubation tube. The gas analyzer 7 is a mainstream gas analyzer measuring gases flowing between the ventilator 2 and the patient 1 without withdrawing samples of the gas to a separate gas analyzer.

The analyzer shown in FIG. 1A is electrically connected via cable 9 to the patient monitor 10. The gas component measured may be carbon dioxide (CO₂), nitrous oxide (N₂O), or any of the volatile anesthetic agents—e.g., halothane, enflurane, isoflurane, desflurane, and sevoflurane. Additionally, there may be a spirometry adapter 11 for measuring the gas flow in the respiratory circuit. In this example, the sensor 12 is located at the distal end of two pressure relying tubes 13. The spirometry sensor may be separately connected as in FIG. 1A or it can be integrated into the mainstream gas analyzer.

In FIG. 1B, a different view of the gas analyzer 7 is depicted to better show the components within the gas analyzer and construction of the adapter 8, which may be disposable or reusable. It is provided with at least one optical window 14 for allowing the IR radiation to be absorbed by the gas components in the measuring chamber between the optical windows. Typically, there are two IR-transmitting optical windows 14. IR emitter 20 is located on one side of the adapter and one or more detector(s) 30 on the opposite side in such a way that the IR radiation is directed from the emitter 20, through the windows 14 and to the detector(s) 30.

The signals, or radiation measurement data, from each detector 30 gets amplified and modified to determine the concentration of the respiratory gas component to be measured. As mentioned above, the measured respiratory gas components can be any IR-absorbing component, such as carbon dioxide, nitrous oxide, or different volatile anesthetic agents. All these gases absorb IR radiation within some specific wavelength region and this region is selected (i.e., the measurement wavelength), such as using a narrowband infrared filter, and provided to the detector 30.

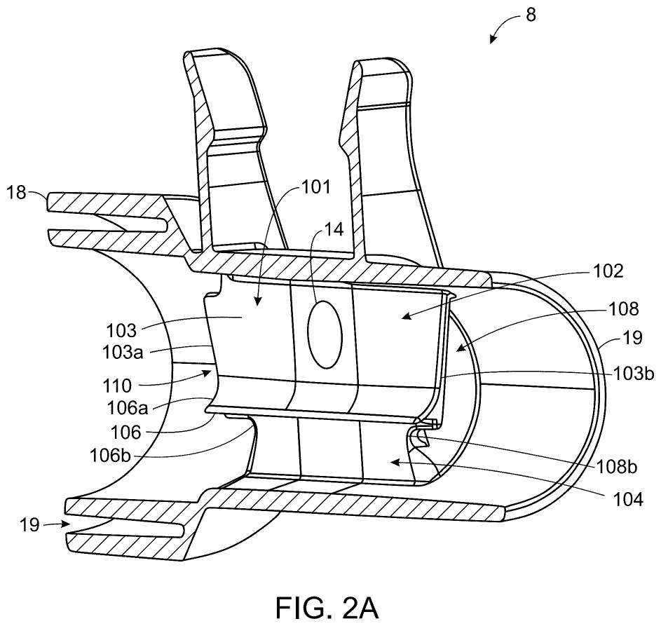

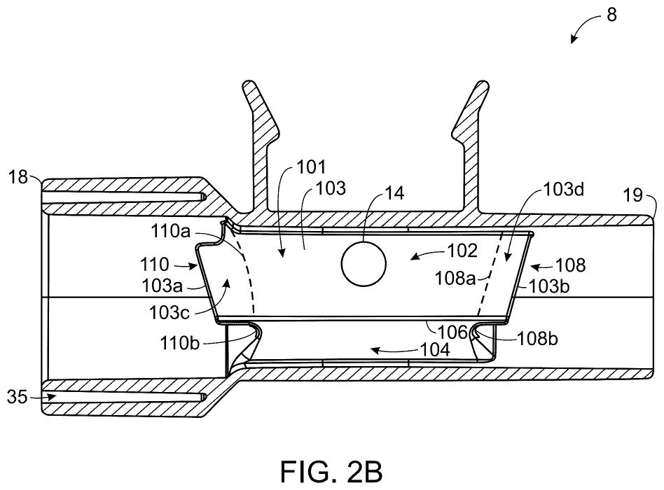

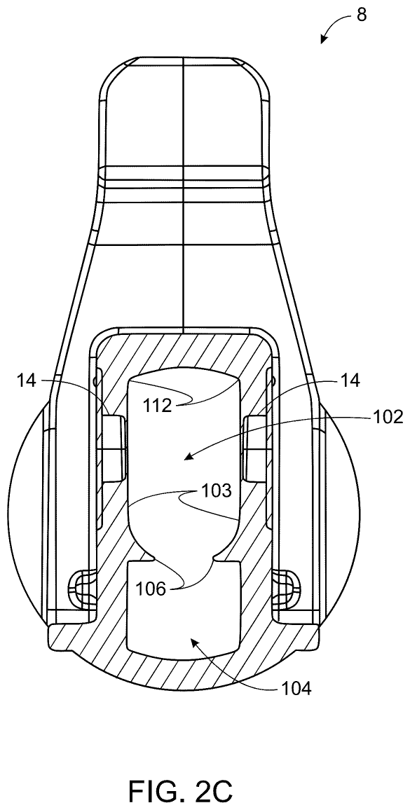

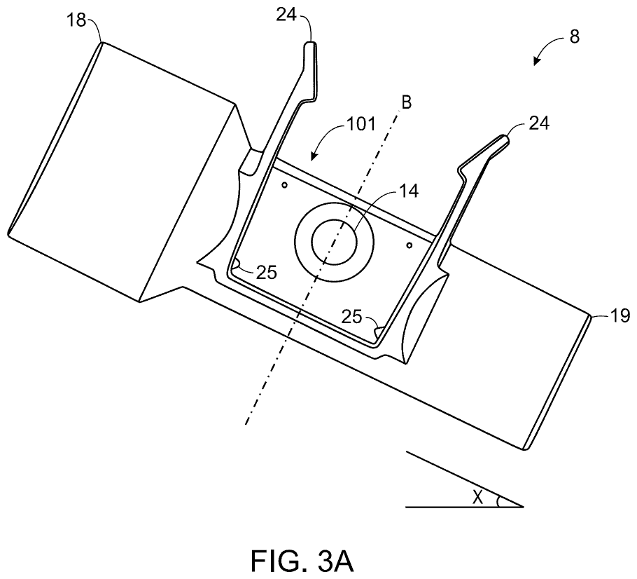

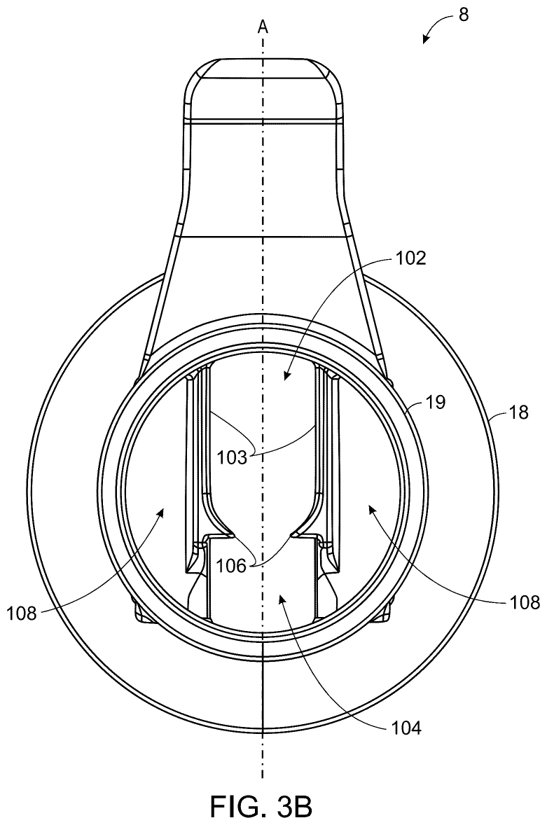

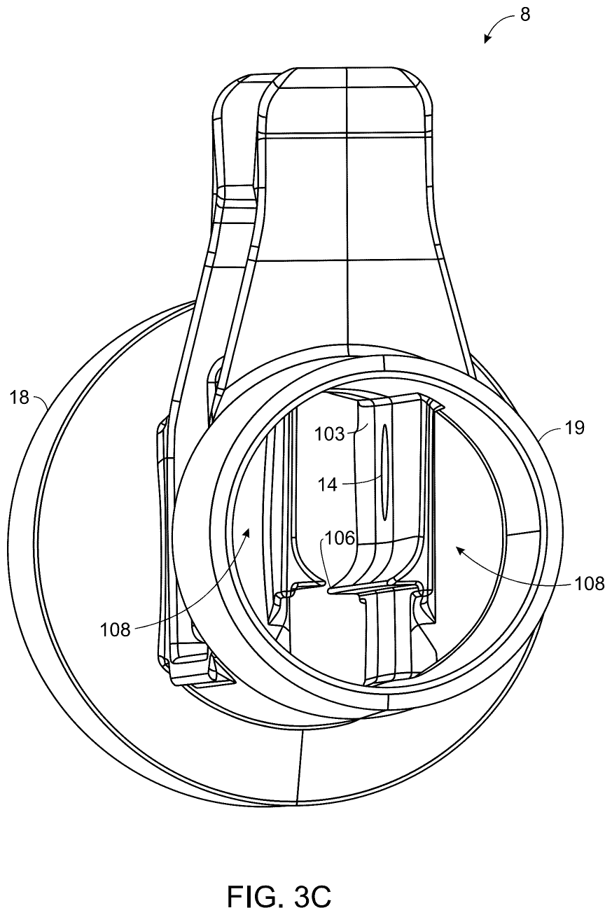

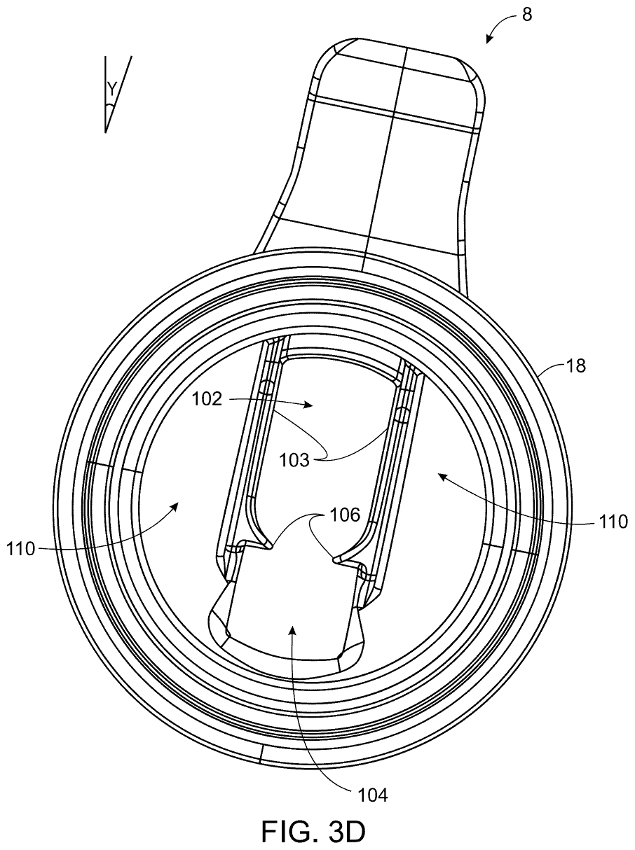

FIGS. 2A and 2B are cross-sectional views of the airway adapter taken along cross-section line A in FIG. 3B, according to an embodiment. In some embodiments, the not-depicted half of the airway adapter of FIGS. 2A and 2B may be a complimentary structure (e.g. mirrored) of the depicted half.

FIG. 2C is a cross-sectional view of the airway adapter taken along cross-section line B in FIG. 3A.

As shown in FIG. 2A, the airway adapter has a narrowed, central portion 101. The outside of the central portion 101 is sized and shaped to accept the gas analyzer 7, as shown in FIG. 1B. The narrowed portion 101 provides an upper measurement chamber 102 and a lower fluid channel 104 within a conduit of the airway adapter 8. An area of the measurement chamber 102 is defined by an upper wall 103 having window 14 provided therein and a dividing protrusion 106. According to an embodiment, at least a portion of an inner surface of the upper wall 103 that includes the window 14 may be flat. Since liquid droplets can accumulate on seams due to the surface tension forces of liquid droplets, the window 14 and the surrounding upper wall 103 may be a molded out of a single piece of material to avoid seams around the window 14. Without seams, liquids droplet accumulation around the window 14 can be decreased. Both of a patient side edge 103a and a ventilator side edge 103b of the upper wall may be angled inward from top to bottom to direct any fluid accumulation into the fluid channel 104 when the airway adapter 8 is in a vertical or near vertical orientation (e.g., rotated 90 degrees from the orientation shown in FIG. 2B).

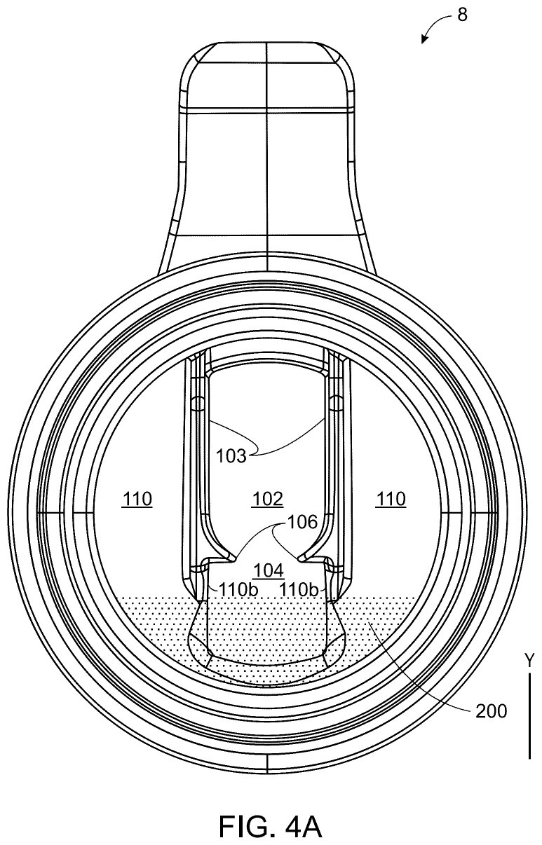

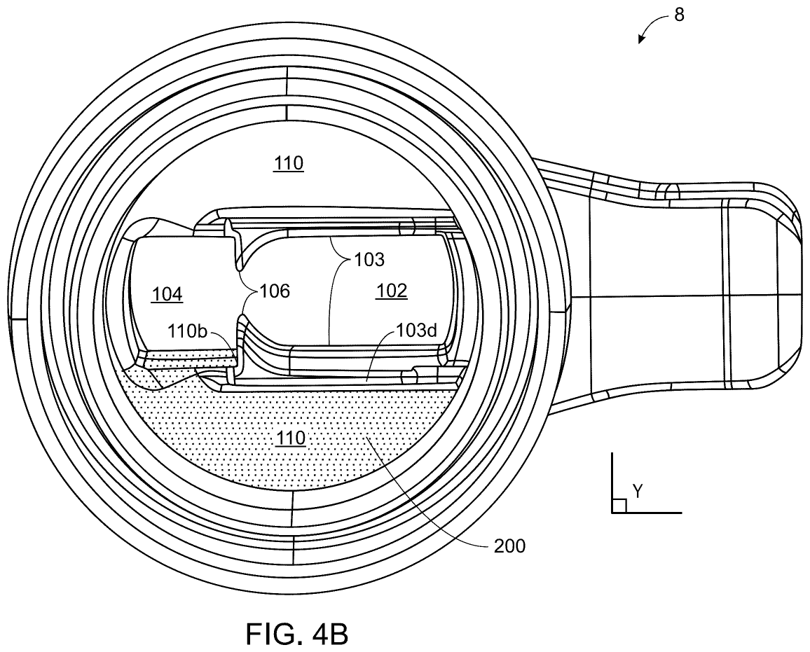

The dividing protrusion 106 may extend across a lower edge of the measurement chamber 102 to divide the measurement chamber 102 and the liquid channel 104. As best seen in FIG. 2C, an upper side 106a of the protrusion 106 gradually tapers outwards while a lower side 106b of the protrusion 106 abruptly extends back to a wall of the airway adapter 8. The gradually tapered upper side of the dividing protrusion 106 guides any liquid droplets down the wall into the fluid channel 104, while the abrupt lower side 106b of dividing protrusion 106 will catch water droplets flowing towards the measurement chamber 102. That is, when the airway adapter 8 is oriented in the position shown in FIG. 2A, a liquid droplet on window 14 will flow smoothly downward along the upper wall 103, over protrusion 106, and into the fluid pathway 102. When the airway adapter 8 is axially rotated 90 degrees from the orientation shown in FIG. 2A (as shown in FIG. 4B) or further, liquid droplets in the fluid channel 104 will be caught in the corner between the lower side of the protrusion and the wall due to surface tension forces.

A fluid directing cavity 110 is provided on the patient side of the narrowed portion 101 and a fluid directing cavity 108 is positioned on the ventilator side of the narrowed portion 101. As shown by dashed lines 108a and 110a, the cavities are provided behind the upper wall 103 with their lower ends opening into the fluid channel 104 through drainage channels 108b and 110b. The fluid directing cavities 108 and 110 are recessed behind the upper wall 103 which has flanges 103c and 103d on its patient side and ventilator side to prevent liquid from flowing out of the recessed cavities 108 and 110 and into the measurement chamber. Fluid flow is directed out of cavities 108 and into the fluid channel 104 through drainage channels 108b and 110b. The drainage channels 108b and 110b may have rounded edges to provide for smooth fluid flow to avoid accumulation of fluid droplets at the edges. As shown best by the dotted lines 108a and 110a in FIG. 2B, the cavities 108 and 110 are angled inward from top to bottom to direct fluid flow into the fluid channel 104 when the airway adapter 8 is in a vertical or near vertical orientation.

As shown best in FIG. 2C, corners 112 between the upper wall 103 and a top surface of the measurement chamber 102 are rounded to avoid fluid accumulation at the corners.

As best shown in FIGS. 2A and 2B, the patient end of the airway adapter 18 includes a circumferential recess 35 for accepting and creating an airtight seal with an endotracheal tube 3 or another component of a ventilation circuit, and the ventilator end 19 may be sized and shaped to create an airtight seal with a Y-piece 4 or similar connector. The end structures shown in FIGS. 2A and 2B are not intended to be limiting. The patient end 18 and the ventilator end 19 of the airway adapter 8 may be any structure capable of providing an airtight seal with a tube or similar closed structure.

According to an embodiment, all or part of an inner surface of the airway adapter 8 may be hydrophobic to avoid fluid adhesion and provide for better fluid flow.

FIG. 3A shows a side view of the airway adapter, according to an embodiment. FIG. 3B shows a ventilator end view of the airway adapter, according to an embodiment. FIG. 3C shows an angled view of a ventilator end of the airway adapter, according to an embodiment. FIG. 3D shows a patient end view of the airway adapter, according to an embodiment.

FIG. 3A shows the airway adapter 8 at longitudinal angle X of approximately 30 degrees. FIG. 3D shows the airway adapter at an axial angle Y of approximately 20 degrees.

As shown in FIGS. 3B-3D, each side of the airway adapter 8 may mirror the opposing side when split along line A. According to an embodiment, a cross section area of the measurement chamber 102 may be in the range of 30% to 60% the cross-sectional area of the ventilator side 19 to ensure adequate airflow. According to an embodiment, a cross section area of the measurement chamber 102 may be in the range of 30% to 60% the cross-sectional area of the patient side 18 to ensure adequate airflow.

The airway adapter 8 is configured to receive and connect to the gas analyzer 7 on the topside via clips 24, as shown in FIGS. 1A and 1B. As shown in FIG. 3A, the clips provide a channel that accepts the gas analyzer 8. Nubs 25 may be provided at the bottom of the channel to further secure the gas analyzer 7 to the airway adapter 8 via a friction lock when the gas analyzer 7 is fully inserted onto the airway adapter 8. According to other embodiment, a gas analyzer may be attached to the airway adapter via other method known in the art.

FIG. 4A shows a patient end view of the airway adapter at an X angle of zero and a Y angle of zero with accumulated liquid, according to an embodiment. FIG. 4B shows a patient end view of the airway adapter at an X angle of 0 and a Y angle of 90 with accumulated liquid, according to an embodiment.

As shown in FIG. 4A, accumulated liquid 200 will be directed into the liquid channel 104 when airway adapter at an X angle of zero and a Y angle of zero. Any liquid 200 that flows into fluid cavities 110 will be directed to the fluid channel 104 through drainage channels 110b. As shown in FIG. 4A, the internal structure of the airway adapter 8 will direct fluids 200 into the liquid channel 104 and away from the measurement chamber 102 through the forces of gravity.

As shown in FIG. 4B, accumulated liquid 200 will be directed into in the liquid channel 104 when airway adapter at an X angle of zero and a Y angle of 90. Any liquid 200 that flows into the fluid cavity 110 will be directed into the fluid channel 104 through drainage channel 110b. Fluid 200 is prevented from entering the measurement chamber 102 by flange 103d which directs fluid into the drainage channel 110b. As shown in FIG. 4B, the internal structure of the airway adapter 8 will direct fluids 200 into the liquid channel 104 and away from the measurement chamber 102 through the forces of gravity. Fluid 200 will be directed into the fluid channel 104 in a similar manner as shown in FIG. 4B when the airway adapter 8 is at an X angle of zero and a Y angle of −90 due to the mirrored structure on the opposing side of the adapter 8.

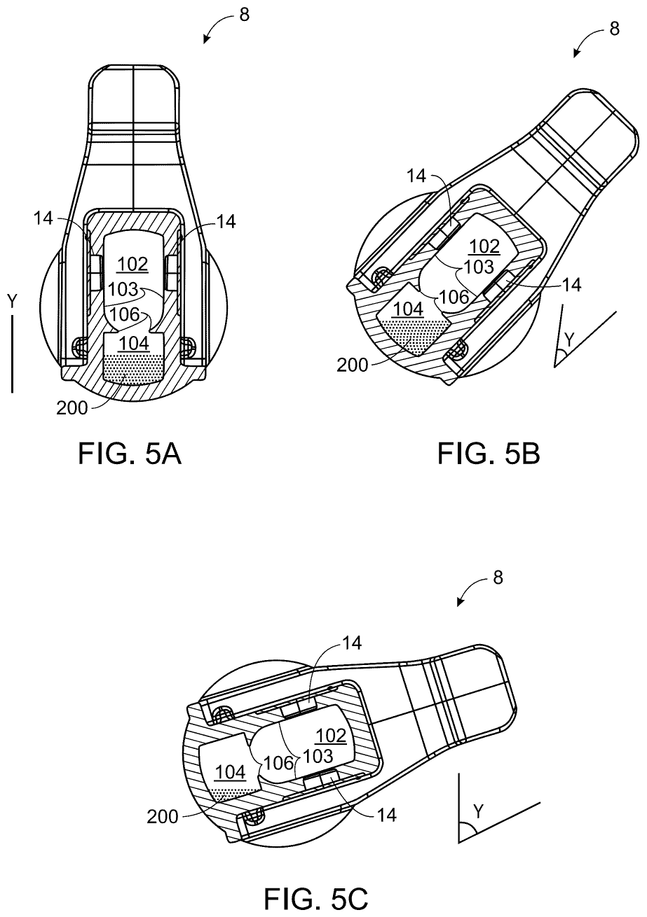

FIG. 5A shows a cross-sectional view of the airway adapter, taken along cross-section line B, at an X angle of zero and a Y angle of zero with accumulated liquid, according to an embodiment. FIG. 5B shows a cross-sectional view of the airway adapter, taken along cross-section line B, at an X angle of zero and a Y angle of 45 with accumulated liquid, according to an embodiment. FIG. 5C shows a cross-sectional view of the airway adapter, taken along cross-section line B, at an X angle of zero and a Y angle of 60 with accumulated liquid, according to an embodiment.

As shown in FIG. 5A, fluid 200 will be directed into the fluid channel 104 by gravity. When the airway adapter 8 is axially rotated to a Y angle of 45 degrees, as shown in FIG. 5B, fluid 200 will be prevented from flowing out of the fluid channel 104 and into the measurement chamber 102 by diverting protrusion 106. Similarly, when the airway adapter 8 is axially rotated to a Y angle of 65 degrees, as shown in FIG. 5C, fluid 200 will be prevented from flowing out of the fluid channel 104 and into the measurement chamber 102 by diverting protrusion 106. A protruding amount of the diversion protrusion may be adjusted based on an estimated amount of liquid. According to an embodiment, the protrusion may protrude in the range of 5% to 25% of into the fluid channel 104 opening. According to an embodiment, a width of the fluid flow channel may be greater than a width of the measurement chamber to provide additional volume for fluid accumulation at high Y angles.

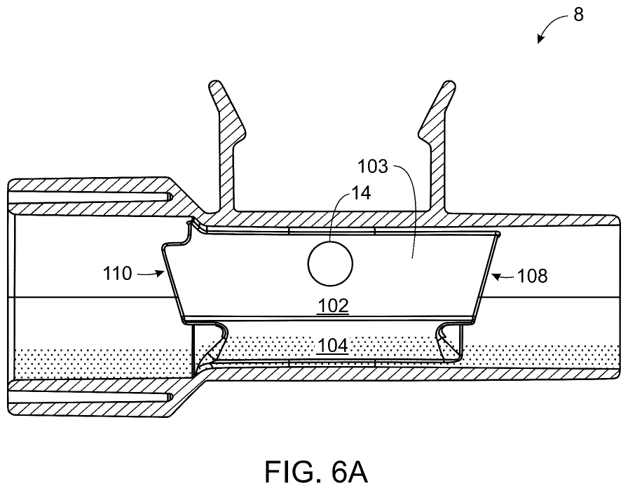

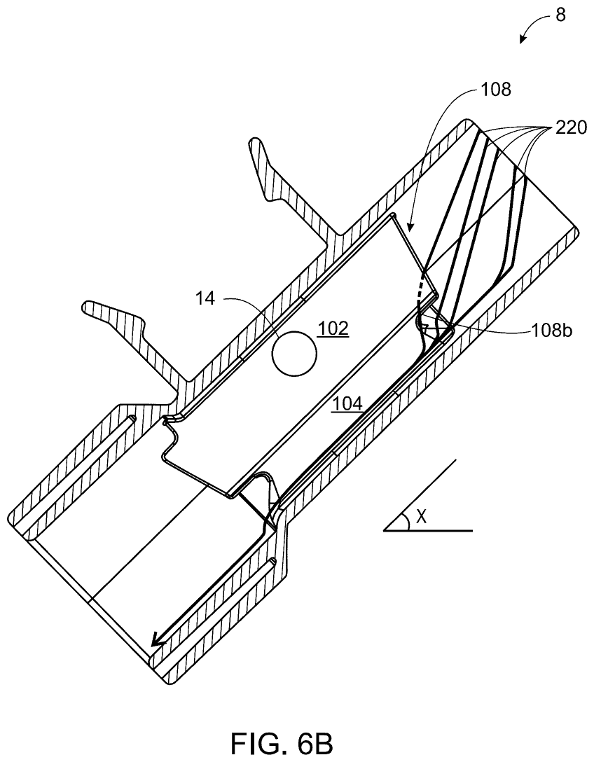

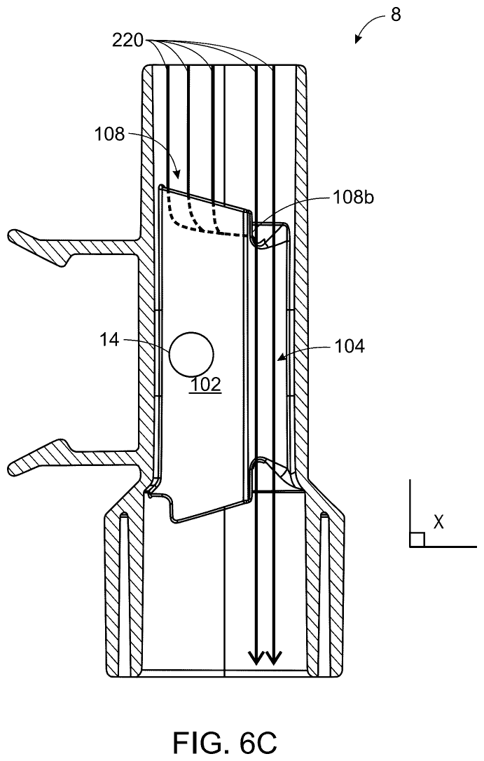

FIG. 6A shows a cross-sectional view of the airway adapter, taken along cross-section line A, at an X angle of zero and a Y angle of zero with accumulated liquid, according to an embodiment. FIG. 6B shows a cross-sectional view of the airway adapter, taken along cross-section line B, at an X angle of 45 and a Y angle of 0 with fluid flow lines, according to an embodiment. FIG. 6C shows a cross-sectional view of the airway adapter, taken along cross-section line B, at an X angle of 90 and a Y angle of 0 with fluid flow lines, according to an embodiment.

As shown in FIG. 6A, when the airway adapter is at an X angle of zero and a Y angle of zero, accumulated liquid flows along a bottom of fluid channel 104. As shown in FIG. 6B, when the airway adapter is at an X angle of 45 and a Y angle of zero, accumulated liquid flows along flow lines 220. The fluid flow lines 220 are directed down into the fluid flow channel 104 by the fluid directing cavity 108 and its drainage channel 108b. As shown more prominently in FIG. 6C, when the airway adapter is at an X angle of 90 and a Y angle of zero, accumulated liquid flows along flow lines 220. The fluid flow lines 220 are diverted by the fluid directing cavity 108 before they can enter the measurement chamber 102. Since the fluid directing cavity 108 slopes downward towards the fluid flow channel 104, the fluid flow lines 220 are directed down into the fluid flow channel 104 through drainage channel 108b. The airway adapter 8 will direct fluid in a similar manner when at an X angle in the range of 0 through −90 by fluid directing cavity 110 and drainage channel 110b.

According to an embodiment, an airway adapter for gas measurement by a mainstream gas analyzer may include a body configured to connect in-series with a ventilation circuit carrying ventilation gas to and from a patient. The body may include a first end, a second end, a top, and a bottom; an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and at least two windows provided in the body at the measurement chamber, the at least two windows configured to pass radiation for measuring gas within the measurement chamber. The inner surface of the airway adapter may be configured to direct fluid away from the measurement chamber and into fluid channel.

According to an embodiment, the airway adapter may include a first ridge protruding out from the inner surface of the body in the narrowed section, the first ridge may span the narrowed section along the flow path of the conduit. An upper edge of the fluid channel may be defined by the first ridge.

According to an embodiment, the airway adapter may further include a second ridge protruding out from the inner surface of the body in the narrowed section, the second ridge may be provided on a wall opposing the first ridge and may span the narrowed section along the flow path of the conduit. An upper edge of the fluid channel may be defined by the first and second ridges.

According to an embodiment, upper facing surfaces of the first and the second ridges may protrude out from the inner surface of the body at a shallower angle than lower facing surfaces of the first and the second ridges.

According to an embodiment, the upper facing surfaces of the first and the second ridges may have a concave shape.

According to an embodiment, an upper end of the narrowed section may be wider along the flow path than a lower end of the narrowed section.

According to an embodiment, inwardly protruding sides of the narrowed section may be concave.

According to an embodiment, the airway adapter may further include a first recessed cavity on the first side of the narrowed section and a second recessed cavity on a second side of the narrowed section.

According to an embodiment, the first and second recessed cavities may extend behind the narrowed section and may be configured to direct fluid flow into the fluid flow channel.

According to an embodiment, the first side of the narrowed section may include a flange that extends over the first recessed cavity and the second side of the narrowed section may include a flange that extends over the second recessed cavity.

According to an embodiment, the first cavity may taper away from the first side as it extends downward and the second cavity may taper away from the second side as it extends downwards.

According to an embodiment, the airway adapter may further include a third recessed cavity on the first side of the narrowed section and a fourth recessed cavity on a second side of the narrowed section, the third and fourth recessed cavities may be on a wall of the airway adapter that is opposite the first and second recessed cavities.

According to an embodiment, a width of the fluid flow channel may be equal to a width of the measurement chamber. A width of the fluid flow channel may be greater than to a width of the measurement chamber.

According to an embodiment, a ventilation system may include a ventilator configured to ventilate a patient; and a ventilation circuit configured to carry ventilation gas from the ventilator the a patient and from the patient to the ventilator, the ventilation circuit comprising and airway adapter configured to connect in-series in the ventilation circuit. The airway adapter may include a body: a first end, a second end, a top, and a bottom, an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and at least two windows provided in the body at the measurement chamber, the at least two windows configured to pass radiation for measuring gas within the measurement chamber. The inner surface of the airway adapter may be configured to direct fluid away from the measurement chamber and into fluid channel.

According to an embodiment, the airway adapter may further include first and second ridges protruding out from the inner surface of the body in the narrowed section, the first and second ridges spanning the narrowed section along the flow path of the conduit,

wherein an upper edge of the fluid channel is defined by the first and second ridges.

According to an embodiment, an upper end of the narrowed section may be wider along the flow path than a lower end of the narrowed section.

According to an embodiment, the airway adapter may further include a first recessed cavity on the first side of the narrowed section and a second recessed cavity on a second side of the narrowed section.

According to an embodiment, the first cavity may taper away from the first side as it extends downward and the second cavity tapers away from the second side as it extends downwards.

According to an embodiment, the airway adapter may further include a third recessed cavity on the first side of the narrowed section and a fourth recessed cavity on a second side of the narrowed section, the third and fourth recessed cavities being on a wall of the airway adapter that is opposite the first and second recessed cavities.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. An airway adapter for gas measurement by a mainstream gas analyzer, the airway adapter comprising:
a body configured to connect in-series with a ventilation circuit carrying ventilation gas to and from a patient, the body comprising:
a first end, a second end, a top, and a bottom;
an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and
at least two windows provided in the body at the measurement chamber, the at least two windows configured to pass radiation for measuring gas within the measurement chamber;
wherein the inner surface of the body is configured to direct fluid away from the measurement chamber and into fluid channel.

2. The airway adapter of claim 1, further comprising a first ridge protruding out from the inner surface of the body in the narrowed section, the first ridge spanning the narrowed section along the flow path of the conduit,
wherein an upper edge of the fluid channel is defined by the first ridge.

3. The airway adapter of claim 2, further comprising a second ridge protruding out from the inner surface of the body in the narrowed section, the second ridge being provided on a wall opposing the first ridge and spanning the narrowed section along the flow path of the conduit,
wherein an upper edge of the fluid channel is defined by the first and second ridges.

4. The airway adapter of claim 3, wherein upper facing surfaces of the first and the second ridges protrude out from the inner surface of the body at a shallower angle than lower facing surfaces of the first and the second ridges.

5. The airway adapter of claim 3, wherein the upper facing surfaces of the first and the second ridges have a concave shape.

6. The airway adapter of claim 1, wherein, an upper end of the narrowed section is wider along the flow path than a lower end of the narrowed section.

7. The airway adapter of claim 6, wherein inwardly protruding sides of the narrowed section are concave.

8. The airway adapter of claim 1, further comprising a first recessed cavity on the first side of the narrowed section and a second recessed cavity on a second side of the narrowed section.

9. The airway adapter of claim 8, wherein the first and second recessed cavities extend behind the narrowed section and are shaped to direct fluid flow into the fluid flow channel.

10. The airway adapter of claim 8, wherein the first side of the narrowed section includes a flange that extends over the first recessed cavity and the second side of the narrowed section includes a flange that extends over the second recessed cavity.

11. The airway adapter of claim 8, wherein the first cavity tapers away from the first side as it extends downward and the second cavity tapers away from the second side as it extends downwards.

12. The airway adapter of claim 8, further comprising a third recessed cavity on the first side of the narrowed section and a fourth recessed cavity on a second side of the narrowed section, the third and fourth recessed cavities being on a wall of the airway adapter that is opposite the first and second recessed cavities.

13. The airway adapter of claim 1, wherein a width of the fluid flow channel is equal to a width of the measurement chamber.

14. The airway adapter of claim 1, wherein a width of the fluid flow channel is greater than to a width of the measurement chamber.

15. A ventilation system comprising:
a ventilator configured to ventilate a patient; and
a ventilation circuit configured to carry ventilation gas from the ventilator to a patient and patient gas from the patient to the ventilator, the ventilation circuit comprising an airway adapter configured to connect in-series in the ventilation circuit, the airway adapter comprising:
a body:
a first end, a second end, a top, and a bottom;
an inner surface providing a conduit which extends through the body to provide a flow path for the ventilation gas to pass between the first end and the second end, wherein opposing sides of a central portion of the inner surface protrude inward to provide a narrowed section, the narrowed section comprising an upper measurement chamber and a lower fluid channel; and
at least two windows provided in the body at the measurement chamber, the at least two windows configured to pass radiation for measuring gas within the measurement chamber;
wherein the inner surface of the body is configured to direct fluid away from the measurement chamber and into fluid channel.

16. The ventilation system of claim 15, wherein the airway adapter further comprises first and second ridges protruding out from the inner surface of the body in the narrowed section, the first and second ridges spanning the narrowed section along the flow path of the conduit, wherein an upper edge of the fluid channel is defined by the first and second ridges.

17. The ventilation system of claim 15, wherein an upper end of the narrowed section is wider along the flow path than a lower end of the narrowed section.

18. The ventilation system of claim 15, wherein the airway adapter further comprises a first recessed cavity on the first side of the narrowed section and a second recessed cavity on a second side of the narrowed section.

19. The ventilation system of claim 18, wherein the first cavity tapers away from the first side as it extends downward and the second cavity tapers away from the second side as it extends downwards.

20. The ventilation system of claim 18, wherein the airway adapter further comprises a third recessed cavity on the first side of the narrowed section and a fourth recessed cavity on a second side of the narrowed section, the third and fourth recessed cavities being on a wall of the airway adapter that is opposite the first and second recessed cavities.
